(19) United States
(12) Patent Application Publication
(10) Pub. No.: US 2026/0345921 A1
(43) Pub. Date: Nov. 5, 2026

(54) SELF-DRIVEN MEDICAMENT CONTAINER AND MEDICAMENT DELIVERY DEVICE

(71) Applicant: SANOFI, Paris (FR)

(72) Inventors: Uwe Dasbach, Frankfurt am Main (DE); Tim Glässer, Russelsheim (DE); Matthias Rau, Russelsheim (DE); Stefan Wendland, Frankfurt am Main (DE)

(21) Appl. No.: 19/490,355

(22) PCT Filed: Jun. 5, 2024
(86) PCT No.: PCT/EP2024/065443

§ 371 Date: Dec. 4, 2025

(30) Foreign Application Priority Data
Jun. 5, 2023 (EP) 23315234.7

(51) Int. Cl. A61M 5/32; A61M 5/20

(52) U.S. Cl. CPC A61M 5/3272; A61M 2005/208

(57) ABSTRACT

A self-driven medicament container is disclosed for storing and dispensing a medicament upon user interaction. The container includes a reservoir body divided by a stopper into a medicament compartment and a drive compartment. The medicament compartment contains the medicament and features an outlet for discharge. A plunger with a shaft incorporates a locking element that engages a retainer to prevent distal movement and a contact element that interfaces with the stopper. A release mechanism disengages the locking element in response to user input, enabling the plunger's distal movement. The drive element, located in the drive compartment, applies axial force to the plunger, moving the stopper and expelling the medicament when unlocked. The stopper seals the medicament compartment from the drive compartment throughout operation. Additionally, a medicament delivery device comprising this self-driven container is provided.

### CROSS-REFERENCE TO RELATED APPLICATIONS

The present application is the national stage entry of International Patent Application No. PCT/EP2024/065443, filed on Jun. 5, 2024, and claims priority to European Application No. EP 23315234.7, filed on Jun. 5, 2023, the disclosures of which are incorporated herein by reference.

### TECHNICAL FIELD

The present disclosure relates to a self-driven medicament container for storing a medicament and discharging said medicament in a dispensing operation in response to a user interaction. The present disclosure further relates to a medicament delivery device, comprising the self-driven medicament container.

### BACKGROUND

Medicament containers storing a medicament usually are used in medicament delivery devices. Conventional medicament containers comprise a medicament compartment containing the medicament and a stopper, which is axially movable relative to a medicament compartment in order to discharge the medicament therefrom. The force for moving the stopper is usually provided by a drive mechanism, for example a pre-tensioned compression spring and a plunger of the medicament delivery device. In an initial state, the medicament delivery device is blocked, such that the plunger cannot be moved relative to the medicament container. In response to one or more user interactions, the medicament delivery device is activated via an activation element in order to unblock the plunger and enable a movement of the plunger relatively to the medicament container. This relative movement is usually transferred to the stopper of the medicament container such that medicament is discharged from the medicament compartment.

Disadvantageously, conventional medicament containers require a medicament delivery device for discharging the medicament, since those containers do not have an own drive mechanism that enables medicament discharge in response to a user interaction. Said drive mechanism needs to be adapted to the specifics of the medicament container that is to be used. This often requires a redesign of several components of the medicament delivery device, which is expensive. Thus, a medicament container having an integrated drive mechanism would be advantageous, such that it can be used in less complex medicament delivery devices.

### SUMMARY

In the following the term “distal” refers to the direction of a medicament container or a medicament delivery device in which medicament is discharged. Accordingly, the term “proximal” refers to the opposite direction. As regards pen-shaped medicament delivery devices, the term “distal” refers to the direction towards the injection site and/or the tip of an injection needle of the device. Accordingly, the term “proximal” refers to the direction pointing away from the injection site and/or the tip of an injection needle of the device.

It is an object of the present disclosure to provide an improved medicament container.

The object is achieved by the subject-matter disclosed herein, for example by the subject-matter defined in the appended independent claims. Advantageous refinements and developments are subject to the dependent claims and/or set forth in the description below.

One aspect of the present disclosure relates to a (self-driven) medicament container for storing and discharging a medicament in response to a user interaction. The medicament container comprises a reservoir body, a retainer, a stopper, a drive element, a plunger and a release mechanism. The stopper is configured to divide an interior of the reservoir body into a drive compartment and a medicament compartment. The medicament compartment is configured to contain the medicament. The stopper may be configured to seal the medicament compartment relative to the drive compartment. The medicament compartment comprises an outlet through which the medicament can be discharged during a dispensing operation. The medicament compartment may be distal to the drive compartment.

The plunger has a shaft with a locking element which is configured to engage the retainer in order to prevent a distal movement of the plunger relative to the reservoir body. For example, the locking element may abut a proximal surface of the retainer, when engaged therewith. The locking element may be formed at a proximal portion of the shaft. The shaft of the plunger may extend along a longitudinal axis of the reservoir body, such that a longitudinal axis of the shaft may be parallel to the longitudinal axis of the reservoir body. For example, the longitudinal axis of the shaft may be parallel to the longitudinal axis of the reservoir body before, during and/or after disengagement of the locking element from the retainer.

The release mechanism is configured to disengage the locking element from the retainer in response to the user interaction, e.g. an activation movement. The disengagement enables a distal movement of the plunger relative to the reservoir body. The retainer may have an opening through which the shaft extends into the drive compartment, e.g. through the retainer.

The drive element is positioned in the drive compartment before and after the locking element is disengaged from the retainer. The drive element is configured to provide an axial force to the plunger in order to move the plunger distally relative to the reservoir body thereby moving the stopper relative to the reservoir body and discharging the medicament, e.g. when the locking element is disengaged from the retainer. The drive element may be part of the medicament container, e.g. completely or at least partially contained in the medicament container, e.g. in the drive compartment of the reservoir body. In particular, the drive element may be at least partially contained in the medicament container before, during and/or after the dispensing operation. As the drive element is part of the medicament container, the medicament container can be designated as “self-driven” as it comprises the drive element and no external drive element is required.

In one embodiment, a distal end of the shaft may be arranged to contact the stopper and transfer a movement of the plunger to the stopper.

In one embodiment, the shaft may further have a contact element configured to contact the stopper and transfer a movement of the plunger to the stopper. The contact element may have a larger cross section than the shaft. The contact element may be formed at a distal end of the shaft.

In one embodiment, the contact element may have a plate-shape, e.g. such that an extension of the contact element in a radial direction is longer, preferably at least twice as long, as an extension of the contact element in the longitudinal direction. The contact element may have a constant cross-section along its longitudinal axis. The cross-section of the contact element may be circular. The longitudinal axis of the contact element may be parallel to the longitudinal axis of the reservoir body. The contact element may be integrally formed with the shaft. Alternatively, the contact element may be connected to the shaft. The connection between the contact element and the shaft may be such that the shaft may not rotate relative to the contact element. The angle formed between the longitudinal axis of the shaft and a proximal surface the contact element may be 90 degrees. The contact element may be formed from a full material.

A distally facing end surface of the contact element may be configured to contact the stopper. The distally facing end surface may be a closed surface. The distally facing end surface may be configured to contact the stopper over its full cross-section. The distally facing end surface may be an even surface. The cross-section of the distally facing end surface may correspond to the cross-section of the contact element, which may be circular. The diameter of the distally facing end surface may almost correspond to a diameter of the stopper. Thus, a contact interface between the contact element and the stopper may be maximized. This may improve force transmission to the stopper and enable an even force distribution over the whole cross-section of the stopper. However, the cross-section of the contact element may be smaller than the cross-section of the stopper, such that the contact element does not contact an inner wall of the reservoir body.

The reservoir body has an interior wall surface that is in contact with the medicament, at least along a length of its medicament compartment. The interior wall surface may define an interior cross-section of the reservoir body. In one embodiment, the interior cross-section of the reservoir body may be constant along the length of the reservoir body along which the stopper is moved during the dispensing operation. In one embodiment, the interior cross-section of the reservoir body may be constant along its whole length. The (shape of the) interior cross-section may be defined by (the shape of) the interior wall surface. The cross-section may be taken perpendicularly relative to the longitudinal axis of the reservoir body. The longitudinal axis may extend between the proximal and distal ends of the container. The interior wall surface of the reservoir body may be configured to be continuously connected to an outer surface of the self-driven medicament container. Thus, there may only be a single wall separating an interior of the reservoir body from the exterior of the reservoir body. In other words, there may be no interspaces when travelling from the interior of the reservoir body to the exterior of the reservoir body or medicament container, just the wall of the reservoir body.

The drive element may be or may comprise a spring, preferably a compression spring. The drive element may surround the shaft. In other embodiments, the drive element may be arranged in the interior of the shaft. The drive element may be formed by more than one spring. The drive element may contact a distal surface of the retainer. The drive element may be positioned between the retainer and the contact element, e.g. between a distal surface of the retainer and a proximal surface of the contact element.

The release mechanism may comprise an activation element, e.g. by an activation movement. The activation movement may comprise an axial and/or rotational movement of the activation element relative to the reservoir body. The axial movement of the activation element may be in a distal movement relative to the reservoir body. The activation movement, which the user may perform, may be converted into movement of the locking element relative to the retainer by interaction between the locking element and the activation element. Due to the relative movement, e.g. between the locking element and the retainer, the self-driven medicament container may be activated.

In one embodiment, the activation element is a push button. The push button may have at least one guide groove. The retainer may have at least one guiding element configured to interact with the guide groove of the push button, when the push button is pushed in a distal direction relative to the reservoir body and/or the retainer and/or the locking element. Due to the interaction of the guide element with the guide groove the push button may be supported by the retainer in a radial direction.

The opening of the retainer may be configured such that the locking element can pass through the retainer in a distal direction, when the locking element is disengaged from the retainer.

A proximal end of the plunger, of the shaft and/or of the locking element may be inside the drive compartment, after the dispensing operation.

In one embodiment the locking element may be formed by two or more flexible arms that are configured to radially deflect, preferably in a radial inward direction, in response to a movement of the activation element relative to the reservoir body, e.g. in response to the activation movement. The flexible arms of the locking element may be evenly distributed in a circumferential direction, such that an angular offset between all flexible arms is the same. Each flexible arm may comprise a radial protrusion, e.g. at a free end, the protrusions being configured to engage with the retainer. The free end may be the proximal end of the arm. Preferably, the protrusion protrudes in a radial outward direction such that the flexible arms are configured to deflect radially inwardly in response to the movement of the activation element relative to the reservoir body.

In one embodiment, the retainer comprises two additional openings. The openings may have a similar cross section as the free ends of the flexible arms such that the free ends of the flexible arms can pass through the retainer when deflected radially outwardly due to a movement of the activation element relative to the reservoir body.

In one embodiment, the self-driven medicament container may comprise an interface which can be established between an engagement feature and an inclined surface. The engagement feature may be formed on one of the locking element and the activation element and the inclined surface may be provided on the other one of the locking element and the activation element. When the interface is established and the engagement feature moves relative to the inclined surface, the locking element may be disengaged from the retainer. In other words, the locking element may be disengaged from the retainer as the movement of the activation element is converted into movement of the locking element via the interface. Preferably, the engagement feature slides along the inclined surface in order to disengage the locking element from the retainer.

In one embodiment, when the engagement feature slides along the inclined surface, the flexible arms are deflected. Preferably, the flexible arms are deflected in a radial inward direction.

In one embodiment, the engagement feature may be a distal end surface of the activation element. The inclined surface may be formed on the flexible arms such that the inclined surface is inclined in a radial inward direction, seen from the distal end of the medicament container.

In one embodiment, the engagement feature may be a proximal end surface of the flexible arms and the inclined surface may be formed at an inner wall surface of the activation element. The inclined surface may be inclined in a radial inward direction, seen from the distal end of the medicament container.

In one embodiment, the engagement feature may be an inclined surface having a different angle of inclination compared to the inclined surface with which it is configured to interact.

In one embodiment, the locking element may be formed by a stopper bar which may be configured to abut a proximal surface of the retainer before the user interaction. The locking element may be configured to rotate relatively to the retainer in order to align with the opening of the retainer in response to the user interaction. The opening is configured to allow the locking element to pass therethrough, when aligned with the opening, e.g. after the user interaction.

In one embodiment, the opening may have a cross-section that is similar to the cross-section of the locking element, such that the locking element can pass the opening in a distal direction after the user interaction. In one embodiment, the opening may have a larger cross-section than the locking element.

The reservoir body may have circular interior cross-section. The diameter of the circular cross-section may be constant, e.g. along the length of the medicament compartment. The outer surface of the reservoir body may form an outer surface of the self-driven medicament container. The outer surface of the reservoir body may define an exterior dimension, e.g. the outer diameter, of the self-driven medicament container.

The retainer may be fixed relative to the reservoir body in an axial direction and/or in a rotational direction. In one embodiment, the retainer may be mounted to a proximal end of the drive compartment. In another embodiment, the retainer may be mounted to an inner wall of the drive compartment. The retainer may be directly connected to the reservoir body, e.g. to a proximal end surface thereof or to an inner wall thereof. Thus, the retainer may form a proximal end of the reservoir body. The connection between the retainer and the reservoir body may be configured to counteract the drive force provided by the drive element.

The self-driven medicament container may be configured for use in an injector housing with an activation mechanism.

The self-driven medicament container may comprise a needle. The needle may be connected to the outlet, such that the medicament can be ejected through the needle. The self-driven medicament container may further comprise a needle shield attachable to the self-driven medicament container, preferably to its distal end. The needle shield may surround the needle such that the needle can be protected against mechanical impacts and kept sterile.

The self-driven medicament container having a needle may comprise a grip, enabling a user to perform a medicament injection without the use of an external device into which the self-driven medicament container is inserted, for example, without being inserted into a medicament delivery device such as an autoinjector.

Another aspect of the present disclosure relates to a medicament delivery device. The medicament delivery device comprises a self-driven medicament container with a needle according to one of the embodiments described in the foregoing. The medicament delivery device is configured to enable a user to inject a medicament stored in the self-driven medicament container through the needle into the user's body. The medicament delivery device further comprises an elongated housing and, optionally, an outer button. The self-driven medicament container abuts the elongated housing. The elongated housing has a distal end wall.

The self-driven medicament container may abut the distal end wall. The distal end wall may provide a distal end stop for the container. The distal end wall may have an opening through which the needle and/or the outlet may protrude distally beyond the housing. In other embodiments, a distal end stop may be proximally offset from the distal end wall of the device. The outer button may be connected to a proximal portion of the housing. The connection may be realized via a snap fit.

The outer button may be configured to interact with the release mechanism of the self-driven medicament container in order to disengage the locking element from the retainer. The outer button may interact with the activation element of the release mechanism.

In one embodiment, the outer button may be connected to the release mechanism such that (e.g. any) movement of the outer button can be directly converted into a corresponding movement of the activation element. In another embodiment, the outer button may be connected to the release mechanism such that a movement of the outer button is transferred into a movement of the activation element in a different spatial direction. For example, a helical movement, i.e. an axial and rotational movement, of the outer button may be transferred into an axial movement of the release mechanism, or vice versa.

In one embodiment, the connection of the outer button and the release mechanism may be realized by friction between a proximal surface of the activation element and a distal surface of the outer button.

In one embodiment, the connection of the outer button and the release mechanism may be realized by an interaction of engagement elements on the outer button and/or the release mechanism.

In one embodiment, the disengagement of the locking element from the retainer may be caused by an axial movement of the outer button relative to the housing.

In one embodiment, the disengagement of the locking element from the retainer may be caused by a rotational movement of the outer button relative to the housing.

In one embodiment, the disengagement of the locking element from the retainer may be caused by a combination of movements of the outer button relative to the housing, for example a combination of an axial movement and a rotational movement, a so called helical movement.

The medicament delivery device may comprise a needle shroud movably connected to the housing. The needle shroud protrudes beyond a tip of the needle, when the needle shroud is in a first position. The needle shroud may be in the first position after the medicament has been ejected from the self-driven medicament container and the medicament delivery device is moved away from an injection site. The needle shroud may be in the first position prior to an activation of the medicament delivery device and/or prior to a penetration of the user's skin by the needle. The first position may be an extended position of the needle shroud.

The needle shroud may be moved proximally relative to the housing to a second position. During the relative proximal movement, an exterior wall surface of the needle shroud or at least a proximal end of the needle shroud may slide along an interior wall surface of the housing. In the second position, the tip of the needle protrudes distally beyond the needle shroud. In the second position, a distal end of the needle shroud may be further proximal than a distal end of the housing. Alternatively, in the second position, the distal end of the needle shroud may be at the same level as the distal end of the housing or further distal than the distal end of the housing. The second position may be a retracted position of the needle shroud.

In one embodiment, the needle shroud may be configured to activate the medicament delivery device, when the needle shroud is moved to its second position. In one embodiment, the needle shroud may activate the medicament delivery device via an interaction with the outer button, when the needle shroud is moved to its second position.

The needle shroud may have an opening through which the needle can pass when the needle shroud is moved proximally relative to the housing from its first position to its second position.

The medicament delivery device may comprise a needle shroud spring. The needle shroud spring may be configured to provide an axial force to the needle shroud. Due to this axial force, the needle shroud may be moved from its second position to its first position in which it extends distally beyond the tip of the needle in order to avoid injury caused by the needle after dispensing operation.

The medicament delivery device may comprise a distal end cap configured to be detachably attached to the housing or to the needle shroud. The distal end cap may be configured to be detached from the housing or the needle shroud by a distal movement relative to the housing or the needle shroud, respectively.

In one embodiment, the distal end cap may be configured to engage with the needle shield of the self-driven medicament device, when the distal end cap is attached to the medicament delivery device. The distal end cap may comprise an engagement groove that fits onto the needle shield, thereby supporting the needle shield in a radial outward direction. The distal end cap may be engaged with the needle shield such that due to the engagement, the needle shield can be removed from the self-driven medicament container, when the distal end cap is removed from the medicament delivery device, for example by a distal movement of the distal end cap relative to the housing.

The housing may comprise a button lock configured to engage with the outer button, for example a lock protrusion thereof, in order to block a distal movement of the outer button relative to the housing when the needle shroud is in its first position. The button lock is configured to disengage from the outer button due to an interaction with the needle shroud, when the needle shroud is moved proximally relative to the housing to its second position.

In one embodiment, the button lock may comprise at least two blocker arms. The blocker arms may be configured to deflect radially outwardly due to an interaction with the needle shroud, when the needle shroud is moved proximally relative to the housing. For example, a proximal end of the needle shroud may slide along the blocker arms thereby deflecting a free end of the blocker arms in a radial direction, when the needle shroud is moved proximally relative to the housing. Due to the radial deflection, the blocker arms may be disengaged from the outer button. This enables a distal movement of the outer button relative to the housing. The blocker arms may deflect radially outwardly or radially inwardly, depending on whether the proximal end of the needle shroud is inside or outside of the blocker arms in a radial direction.

The needle shroud spring may be positioned between the housing and the needle shroud. Preferably, the needle shroud spring may be positioned between the distal end wall of the housing and the needle shroud. More preferably, the needle shroud spring may be positioned between a distal surface of the distal end wall of the housing and a proximal inner surface of the needle shroud.

The housing may comprise a protrusion at its distal end, which extends the housing in a distal direction. The protrusion may be configured to connect the distal end cap to the housing and/or axially guide a movement of the distal end cap during attachment to or detachment from the housing.

The housing may have a circular interior cross-section with at least along a part of its length. Where the housing has a circular interior cross-section, the diameter of the housing may be constant.

In one embodiment, the medicament delivery device may be an autoinjector.

In one embodiment, the medicament delivery device may comprise a guide unit, preferably attached to the housing. The needle shroud may comprise a guide pin, a torsion protection and/or flexible bars. The guide pin may protrude from an outer wall of the needle shroud outwardly. The flexible bars may be separated by through-recesses extending through the wall of the needle shroud. The flexible bars and a body of the needle shroud may be made of one piece. The torsion protection may comprise a bar, preferably extending in an axial direction of the needle shroud.

The guide unit may comprise a first channel and a second channel. The first channel may extend firstly with a slide inclination against the axial direction and then basically in the axial direction towards a bent of the first channel and then back towards a dead end of the first channel. The extension towards the dead end may comprise a slide inclination against the axial direction and/or may be parallel to the axial direction. The dead end may be separated from the rest of the first channel by a barb.

In a first state of the needle shroud, the guide pin may be arranged in a part of the first channel below the dead end and at the beginning of the inclination of the first channel.

The torsion protection of the needle shroud may be arranged within the second channel. The torsion protection may be guided by the second channel during the movement of the needle shroud relative to the guide unit. The second channel may be straight and parallel to the moving direction of the needle shroud. The torsion protection within the second channel may serve as a protection of the needle shroud against a rotation of the needle shroud.

In a second state of the needle shroud, the needle shroud may be partly arranged within the guide unit, e.g. because of the medicament delivery device being partly arranged on the skin of the user. In the second state of the needle shroud, the guide pin may be moved within the first channel towards the bent. When the guide pin passes the inclination of the first channel, an upper portion of the needle shroud may be moved perpendicular to the moving direction of the needle shroud and the flexible bars may be flexed, because the rest of the needle shroud is secured against any rotation by the torsion protection within the second channel. Then, the flexible bars are biased.

In a third state of the needle shroud, the needle shroud may be pressed into the guide unit completely, e.g. because of the user arranging the medicament delivery device on his/her skin. So, in the third state of the needle shroud, the needle may be exposed by the needle shroud. In this situation, the guide pin has arrived in the bent of the first channel and may be moved perpendicular to the moving direction of the needle shroud within the bent. In the third state of the needle shroud, the biased flexible bars may force the guide pin through the bent.

In a fourth state of the needle shroud, the flexible bars may be released and the guide pin may have moved perpendicular to the moving direction of the needle shroud within the bent.

In a fifth state of the needle shroud, the medicament delivery device may be partly removed from the skin of the user. The guide pin may be forced over the barb of the first channel such that the flexible bars may be biased again. When the medicament delivery device is removed from the skin of the user, the needle shroud may be pushed out of the housing in a distal direction, for example by the needle shroud spring such that the guide pin may be forced over the barb.

In a sixth state of the needle shroud, the medicament delivery device may be completely removed from the skin of the user. The needle shroud may completely cover the needle. The guide pin may snap into the dead end of the first channel such that the needle shroud may be fixedly engaged to the guide unit.

We note that features described above and below in conjunction with different embodiments or aspects can be combined with one another, even if such a combination is not explicitly disclosed herein above or below. Further features, advantages and expediencies of the disclosure and, particularly, of the proposed concepts will become apparent from the following description of the exemplary embodiments in conjunction with the drawings.

The self-driven medicament container according to the present disclosure provides the advantage that medicament stored therein can be discharged without the need of an external drive element or delivery device. Thus, the self-driven medicament container can be used as a standalone device.

When used in a medicament delivery device, for example an autoinjector, the self-driven medicament container provides the advantage that the structure of the medicament delivery device can be simpler compared to conventional medicament delivery devices. For example, the medicament delivery device does not require a drive mechanism or a drive element. Thus, the medicament delivery device can be less complex and has less risk of failure. In addition, the medicament delivery device can be used with self-driven medicament containers of the same or different lengths without requiring a structural adaption. Thus, costs can be saved and waste can be reduced. If, nonetheless, a structural adaptation is necessary, less components of the medicament delivery device need to be adapted, which simplifies the redesign process and reduces costs.

The medicament delivery device according to the present disclosure provides the advantage that it has a simpler structure than conventional medicament delivery devices since it does not require a drive mechanism or drive element. Accordingly, a redesign might not always be necessary even when self-driven medicament containers with different geometries are to be used therein. If, nonetheless, a redesign becomes necessary, less components of the medicament delivery device need to be adapted, which simplifies the redesign process and reduces costs.

### BRIEF DESCRIPTION OF THE DRAWINGS

FIG. 1 illustrates an embodiment of a self-driven medicament container prior to a dispensing operation.

FIG. 2 illustrates an embodiment of a self-driven medicament container at the beginning of the dispensing operation, after having been activated by a user interaction.

FIG. 3 illustrates an embodiment of a self-driven medicament container at the end of the dispensing operation.

FIG. 4 illustrates another embodiment of a self-driven medicament container prior to a dispensing operation.

FIGS. 5A and 5B illustrate an embodiment of a locking element of a self-driven medicament container.

FIGS. 6A and 6B illustrate embodiments of the self-driven medicament containers of FIGS. 1 to 3 and 4 to 5, respectively, comprising a needle.

FIG. 7 illustrates an embodiment of a medicament delivery device prior to an activation by a user.

FIGS. 8A to 8E illustrate different states of the medicament delivery device of FIG. 7 during a dispensing operation.

FIG. 9 illustrates an exemplary embodiment of a needle shroud and a guide unit in a first state.

FIG. 10 illustrates the needle shroud and the guide unit of FIG. 9 in a second state.

FIG. 11 illustrates the needle shroud and the guide unit of FIG. 9 in a third state.

FIG. 12 illustrates the needle shroud and the guide unit of FIG. 9 in a fourth state.

FIG. 13 illustrates the needle shroud and the guide unit of FIG. 9 in a fifth state.

FIG. 14 illustrates the needle shroud and the guide unit of FIG. 9 in a sixth state.

FIG. 15 illustrates an expanded structural formula, molecular formula, and molecular weight of fitusiran.

Identical elements, elements of the same kind and identically or similarly acting elements may be provided with the same reference numerals in the figures.

### DETAILED DESCRIPTION

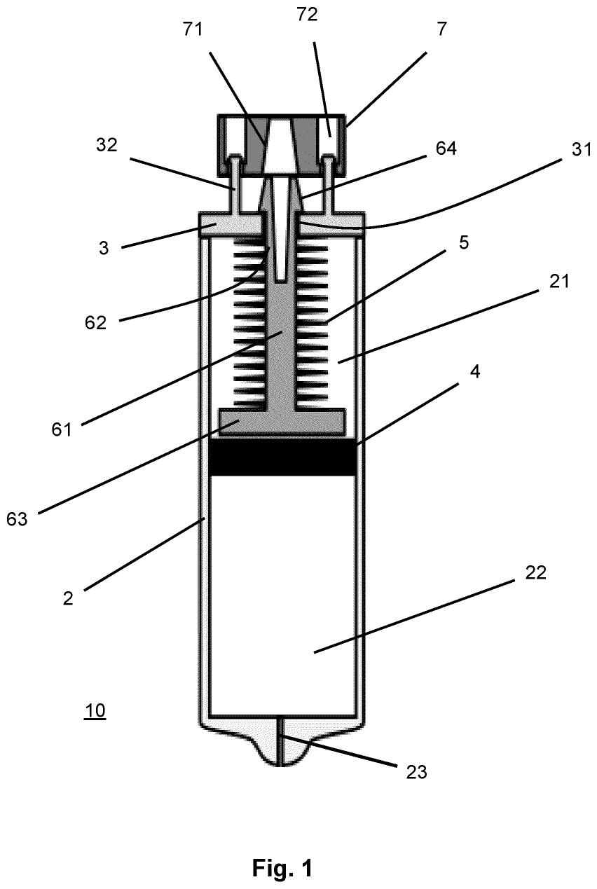

FIG. 1 illustrates an embodiment of a self-driven medicament container 10 according to an aspect of the disclosure. The self-driven medicament container 10 comprises a reservoir body 2, a retainer 3, a stopper 4, a drive element 5, a plunger and a release mechanism. The stopper 4 divides an interior of the reservoir body 2 into a drive compartment 21 and a medicament compartment 22. The medicament compartment 22 contains the medicament. The medicament compartment 22 comprises an outlet 23 through which the medicament can be discharged in a dispensing operation. The stopper 4 seals the medicament compartment 22 towards the drive compartment 21.

The retainer 3 is attached to a proximal end of the drive compartment 22 and forms a proximal end wall of said drive compartment 22. The retainer 3 has an opening 31.

The plunger has a shaft 61 with a locking element at its proximal end. The locking element is formed by two flexible arms 62, each having a radial protrusion at its free end. The protrusion of each flexible arm 62 engages a proximal surface of the retainer 3 thereby preventing a distal movement of the plunger relative to the reservoir body 2.

A contact element 63 is provided at a distal end of the shaft 61 in order to contact the stopper 4 and transfer a movement of the plunger to the stopper 4. The contact element 63 has a larger diameter than the shaft 61.

The drive element 5 is fully inside the drive compartment 21 and extends between a distal surface of the retainer 3 and a proximal surface of the contact element 63. The drive element surrounds the shaft 61.

The release mechanism comprises an activation element formed as a push button 7. The push button 7 has an engagement feature, in particular an inclined surface 71 that interacts with inclined surfaces 64 of the flexible arms 62, when the push button 7 is moved in a distal direction relative to the reservoir body 2, the retainer 3 and/or the plunger. The push button 7 further has guide grooves 72 that interact with guide elements 32 of the retainer 3, thereby radially supporting the push button 7 during its distal movement. The inclined surface 71 has a different angle of inclination than the inclined surfaces 64.

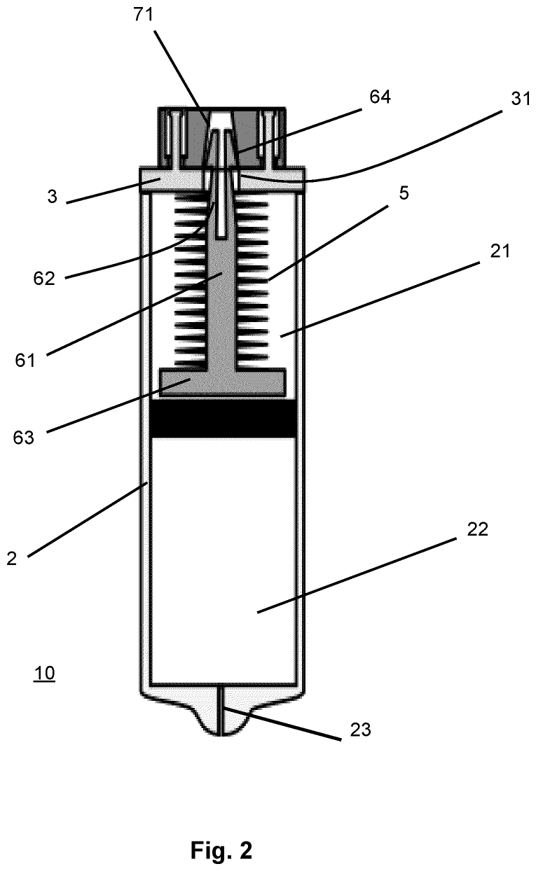

As illustrated in FIG. 2, due to the interaction of the inclined surface 71 and the inclined surfaces 64, the flexible arms 62 deflect radially inwardly such that the free ends of the flexible arms 62 disengage from the proximal surface of the retainer 3. This enables a distal movement of the plunger relative to the reservoir body 2.

Due to a force provided by the drive element 5, the plunger is moved distally and the free ends of the flexible arms 62 pass through the opening 31 of the retainer 3 (not shown in FIG. 2). The distal movement of the plunger is transferred to the stopper 4 which moves distally relative to the reservoir body 2 thereby dispensing medicament stored in the medicament compartment 22 through the outlet 23.

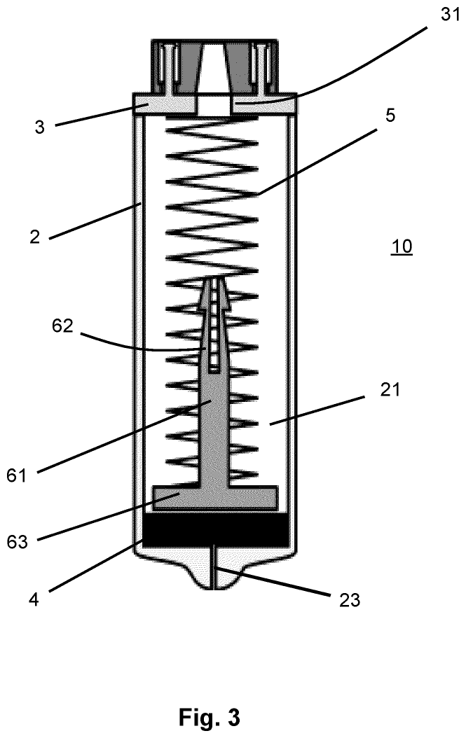

FIG. 3 illustrates the self-driven medicament container 10 at the end of the dispensing operation. The stopper 4 has reached a final distal position at a distal end of the reservoir body 2 and all medicament stored in the medicament compartment 22 has been discharged through the outlet 23. The free ends of the flexible arms 62 are fully inside the drive compartment 21 after having passed through the opening 31 of the retainer 3.

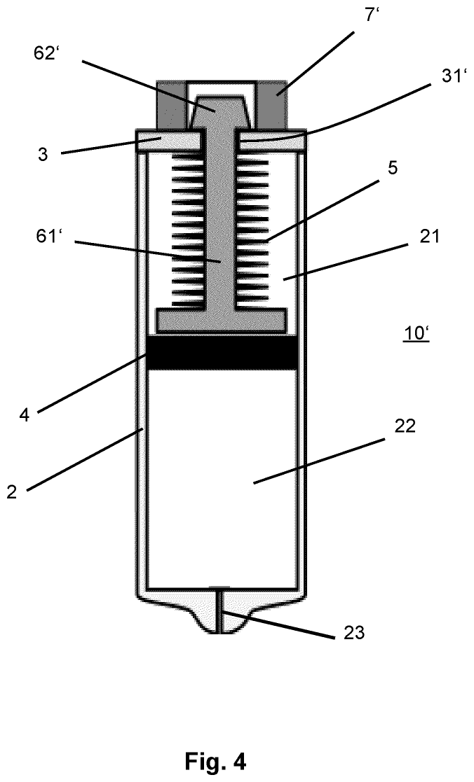

FIG. 4 illustrates another embodiment of a self-driven medicament container 10′. The shaft 61′ of the plunger has a locking element formed as a stopper bar 62′. The stopper bar 62′ abuts a proximal surface of the retainer 3, thereby preventing a distal movement of the plunger relative to the reservoir body 2. Accordingly, the release mechanism has an activation element 7′ that is adapted to cause a rotation of the stopper bar 62′ relative to the retainer 3. The activation element 7′ may be connected to the stopper bar 62′ in a rotationally fixed manner or in a manner suitable to cause a rotational movement of the stopper bar 62′ relative to the retainer 3 in response to a user interaction. Due to the rotational movement of the stopper bar 62′ relative to the retainer 3, a cross-section of the stopper bar 62′ may align with an opening 31′ (not shown) of the retainer 3, as described in the following, thereby allowing a movement of the plunger relative to the reservoir body 2.

The structure and the other components of the self-driven medicament container of FIG. 4, even if not all labeled in detail, correspond to the structure and components of the self-driven medicament container as illustrated in FIGS. 1 to 3.

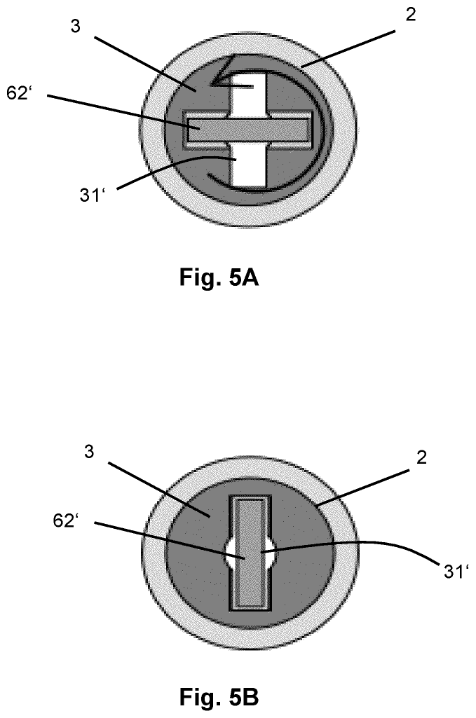

As illustrated in FIG. 5A, the shape of the opening 31′ of the retainer 3 is adapted to the shape of the stopper bar 62′. Due to a rotation of the stopper bar 62′ relative to the retainer 3, as exemplarily indicated by the arrow, the stopper bar 62′ aligns with the opening 31′. This leads to a disengagement of the stopper bar 62′ from the retainer 3. FIG. 5B illustrates the stopper bar 62′ aligned with the opening 31′. In this position, the stopper bar 62′ can pass through the opening 31′, thereby enabling a distal movement of the plunger relative to the reservoir body 2 (not shown).

The dispensing operation and interaction of components after disengagement of the stopper bar 62′ from the proximal surface of the retainer 3 is similar to the dispensing operation described with respect to FIGS. 1 to 3. At the end of the dispensing operation, the stopper bar 62′ is fully inside the drive compartment 21.

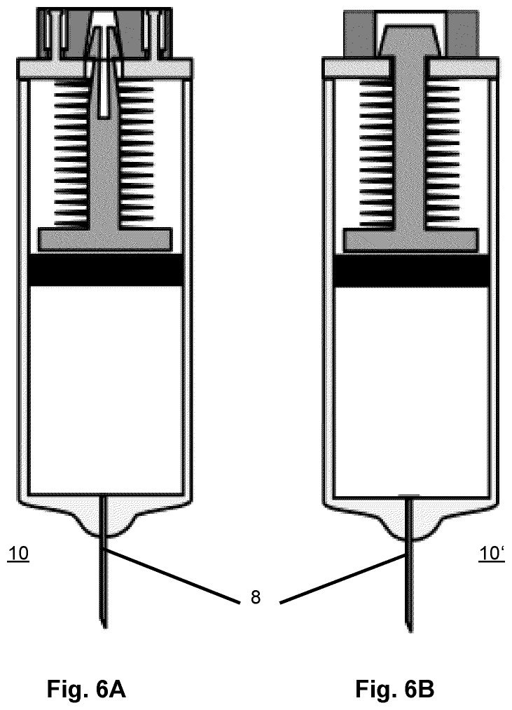

FIGS. 6A and 6B illustrate embodiments of a self-driven medicament container according to the illustrations in FIGS. 1 to 3 and FIGS. 4 to 5, respectively. The self-driven medicament container of FIGS. 6a and 6B additionally comprises a needle 8 such that a user can inject and dispense the medicament from the self-driven medicament container without an additional injection device. The needle is protected and kept sterile by a needle shield 24 (not shown) attachable to the distal end of the reservoir body 2.

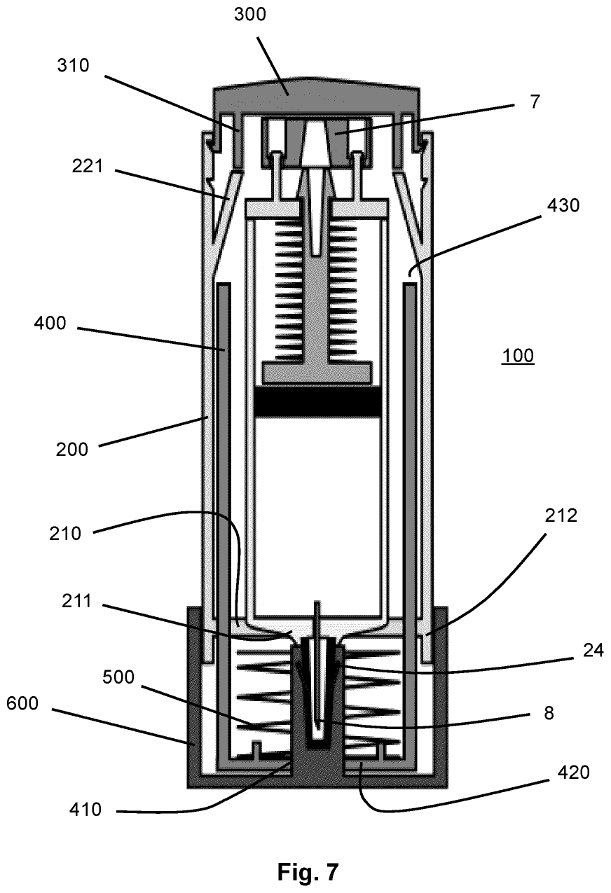

FIG. 7 illustrates an embodiment of a medicament delivery device 100 according to an aspect of the disclosure. The medicament delivery device 100 comprises the self-driven medicament container 10 illustrated in FIGS. 1 to 3 and 6A. The medicament delivery device 100 comprises an elongated housing 200 and an outer button 300. The elongated housing 200 has a distal end wall 210 on which the self-driven medicament container 10 abuts. The distal end wall 210 has an opening 211 through which a needle 8 protrudes distally beyond the housing 200. The outer button 300 is connected to a proximal portion of the housing 200. A distal inner surface of the outer button 300 abuts a proximal surface of the activation button 7, such that a distal movement of the outer button 300 is transferred to the activation button 7.

The medicament delivery device 100 further comprises a needle shroud 400 movably connected to the housing 200. The needle shroud 400 protrudes beyond a distal end of the housing 200 and a tip of the needle 8, when the needle shroud 400 is in a first position, i.e. a position prior to an activation of the medicament delivery device 100 and prior to a penetration of the user's skin. The needle shroud 400 has an opening 410 through which the needle 8 passes when the needle shroud 400 is moved proximally relative to the housing 200.

The medicament delivery device 100 comprises a needle shroud spring 500 positioned between the distal end wall 210 and a proximal inner surface 420 of the needle shroud 400. The needle shroud spring 500 provides distal force to the needle shroud 400 when it is compressed by a proximal movement of the needle shroud 400 relative to the housing 200.

The medicament delivery device 100 comprises a distal end cap 600 configured be detachably attached to the housing 200. The distal end cap 600 comprises an engagement groove that fits over the needle shield 24, thereby supporting the needle shield 24 in a radial outward direction. By a distal movement relative to the housing 200, the distal end cap 600 may be removed from the housing 200 by a user. Due to the engagement with the distal end cap 600, the needle shield 24 may be removed from the reservoir body 2, when the distal end cap 600 is removed from the housing 200.

The housing 200 further comprises a button lock. The button lock comprises two blocker arms 221 configured to engage with a lock protrusion 310 of the outer button 300 thereby blocking a distal movement of the outer button 300 relative to the housing 200 when the needle shroud 400 is in its first position. The blocker arms 221 are flexible and configured to deflect radially outwardly due to an interaction with a proximal end 430 of the needle shroud 400, when the needle shroud 400 is moved proximally relative to the housing 200. Due to the radial outward deflection, the blocker arms 221 are disengaged from the lock protrusion 310. This enables a distal movement of the outer button 300 relative to the housing 200.

he housing 200 comprises a protrusion 212 at its distal end, which extends the housing in a distal direction. The protrusion 212 is configured to connect the distal end cap 600 to the housing and axially guide a movement of the distal end cap 600 when it is attached to or detached from the housing 200.

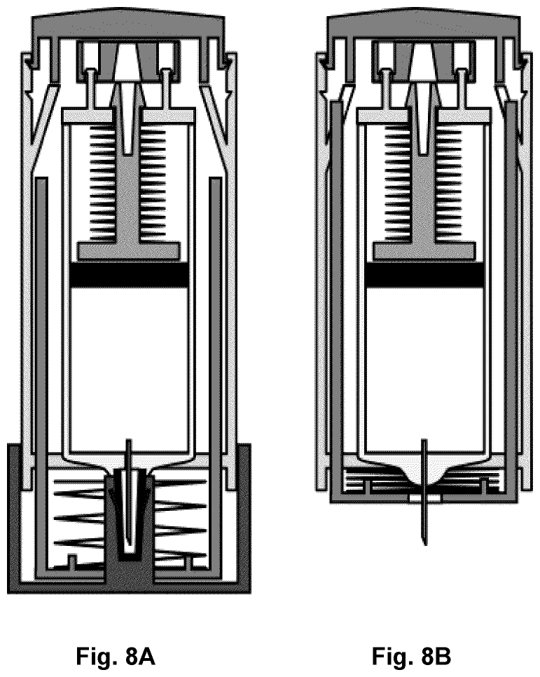

FIGS. 8A to 8E illustrate different states of the medicament delivery device 100 during a dispensing operation. In FIG. 8A, the medicament delivery device is in an initial position. The self-driven medicament container is inserted into the housing of the medicament delivery device. The end cap is still attached to the housing and the needle shroud is in a first position. FIG. 8B illustrates the start of the dispensing operation. The cap has been removed and the user may press the distal end of the needle shroud against the desired injection site such that the needle shroud may move proximally to its second position inside the housing and the needle can penetrate the user's skin (not shown). Due to the proximal movement of the needle shroud relative to the housing the needle shroud spring is compressed.

As illustrated in FIG. 8C, the user then may activate the medicament delivery device by pushing the outer button distally relative to the housing. This disengages the locking element of the self-driven medicament container from the retainer. Due to the force of the drive element in the drive compartment, the plunger and the stopper are pushed distally relative to the reservoir body. Due to the distal movement of the stopper relative to the reservoir body, the medicament stored in the medicament compartment is discharged through the outlet and the needle. In other words, the medicament is injected into the user's body.

FIG. 8D illustrates the end of the dispensing operation. The stopper has reached the distal end wall of the reservoir body. Thus, the medicament compartment has been emptied and all medicament has been injected into the user's body.

As illustrated in FIG. 8E, when the user removes the medicament delivery device from the injection site, the force of the needle shroud spring pushes the needle shroud distally relative to the housing and the needle so that the tip of the needle is covered by the needle shroud.

Any invention described herein is not limited by the description in conjunction with the exemplary embodiments. Rather, the invention and the associated disclosure comprise any new feature as well as any combination of features, particularly including any combination of features in the patent claims, even if said feature or said combination per se is not explicitly stated in the patent claims or exemplary embodiments.

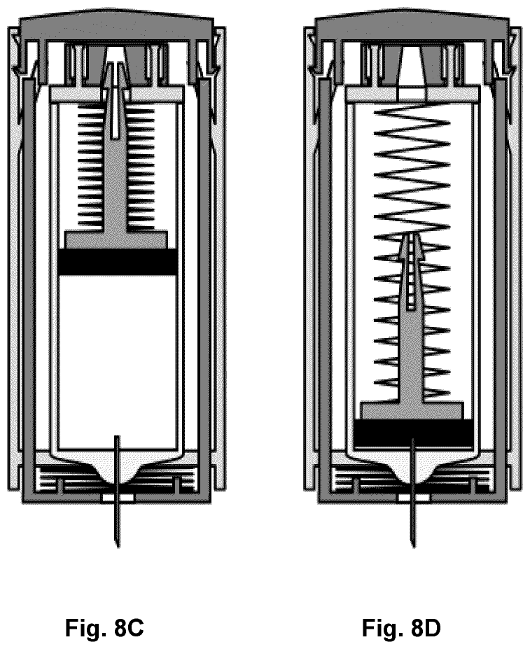

FIG. 9 illustrates an exemplary embodiment of a needle shroud 400 and a cutaway side view of a guide unit 700 in a first state. In the first state of the needle shroud 400, the medicament delivery device 100 is not yet arranged on the skin of the user and the needle shroud 400 protects the needle 8.

The needle shroud 400 comprises a guide pin 90, a torsion protection 92, and flexible bars 94. The guide pin 90 protrudes from an outer wall of the needle shroud 400 outwardly. The flexible bars 94 are separated by through-recesses extending through the wall of the needle shroud 400. So, the flexible bars 94 and a body of the needle shroud 400 may be made of one piece. The torsion protection 92 may comprise a bar extending in an axial direction of the needle shroud, i.e. vertically in FIG. 9.

The guide unit 700 comprises a first channel 96 and a second channel 97. The first channel 96 extends firstly with a slide inclination against the axial direction and then basically in an axial direction towards a bent 98 of the first channel 96 and then back towards a dead end 99 of the first channel 96. The dead end 99 is separated from the rest of the first channel 96 by a barb 102. In the first state of the needle shroud 400, the guide pin 90 is arranged in a part of the first channel 96 below the dead end 99 and at the beginning of the inclination of the first channel 96.

The torsion protection 92 of the needle shroud 400 is arranged within the second channel 97 and is guided by the second channel 97 during the movement of the needle shroud 400 relative to the guide unit 700. The second channel 97 is straight and parallel to the moving direction of the needle shroud 400. The torsion protection 92 within the second channel 97 serves as a protection of the needle shroud 400 against a rotation of the needle shroud 400.

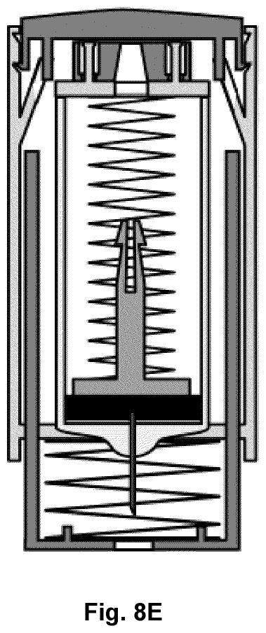

FIG. 10 illustrates the needle shroud 400 and the guide unit 700 of FIG. 9 in a second state. In the second state of the needle shroud 400, the needle shroud 400 may be partly arranged within the guide unit 700, e.g. because of the medicament delivery device 100 being partly arranged on the skin of the user. In the second state of the needle shroud 400, the guide pin 90 is moved within the first channel 96 towards the bent 98. When the guide pin 90 passes the inclination of the first channel 96, an upper portion of the needle shroud 400 is moved perpendicular to the moving direction of the needle shroud 400 and the flexible bars 94 are flexed, because the rest of the needle shroud 400 is secured against a rotation by the torsion protection 92 within the second channel 97. Thus, the flexible bars 94 are biased.

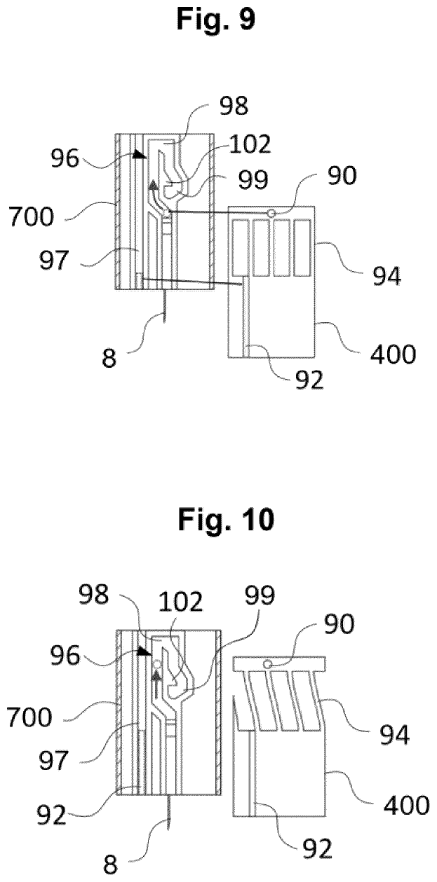

FIG. 11 illustrates the needle shroud 400 and the guide unit 700 of FIG. 9 in a third state. In the third state of the needle shroud 400, the needle shroud 400 is pressed into the guide unit 700 completely, e.g. because of the user arranging the medicament delivery device 100 on his/her skin. So, in the third state of the needle shroud 400, the needle 8 is exposed by the needle shroud 400. In this situation, the guide pin 90 has arrived in the bent 98 of the first channel 96 and may be moved perpendicular to the moving direction of the needle shroud 400 within the bent 98. In the third state of the needle shroud 400, the biased flexible bars 94 force the guide pin 90 through the bent 98.

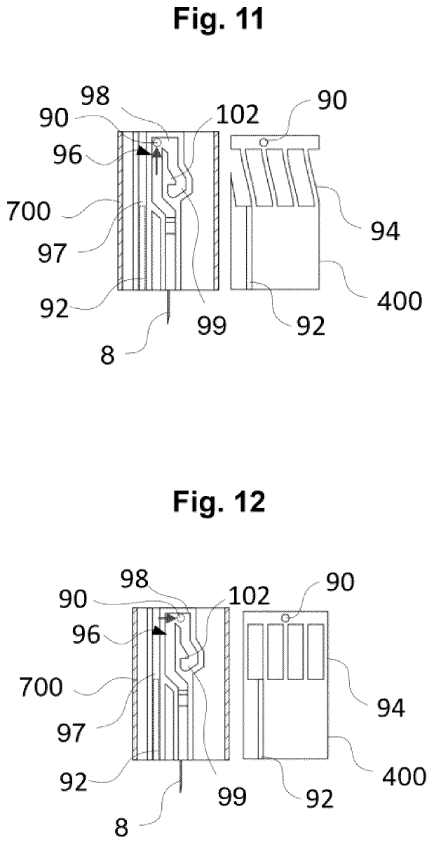

FIG. 12 illustrates the needle shroud 400 and the guide unit 700 of FIG. 9 in a fourth state. In the fourth state of the needle shroud 400, the flexible bars 94 are released and the guide pin 90 has moved perpendicular to the moving direction of the needle shroud 400 within the bent 98.

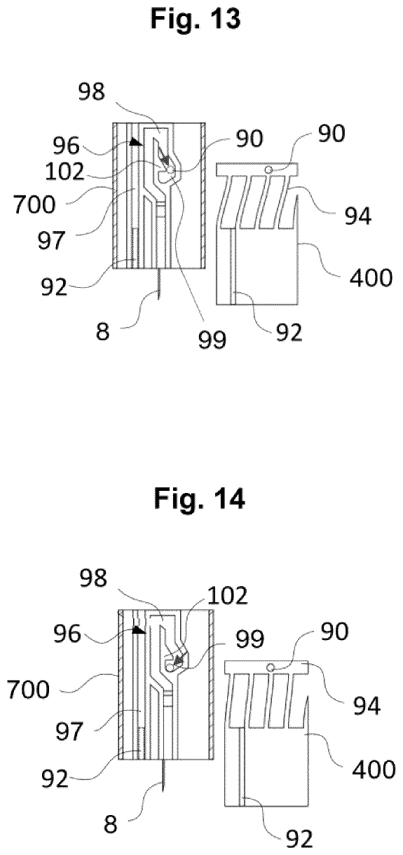

FIG. 13 illustrates the needle shroud 400 and the guide unit 700 of FIG. 9 in a fifth state. In the fifth state of the needle shroud 400, the medicament delivery device 100 may be partly removed from the skin of the user. The guide pin 90 is forced over the barb 102 of the first channel 100 such that the flexible bars 94 are biased again. When the medicament delivery device 100 is removed from the skin of the user, the needle shroud 400 may be pushed out of the housing 200, for example by a conventional needle shroud spring (not shown) such that the guide pin 90 is forced over the barb 102.

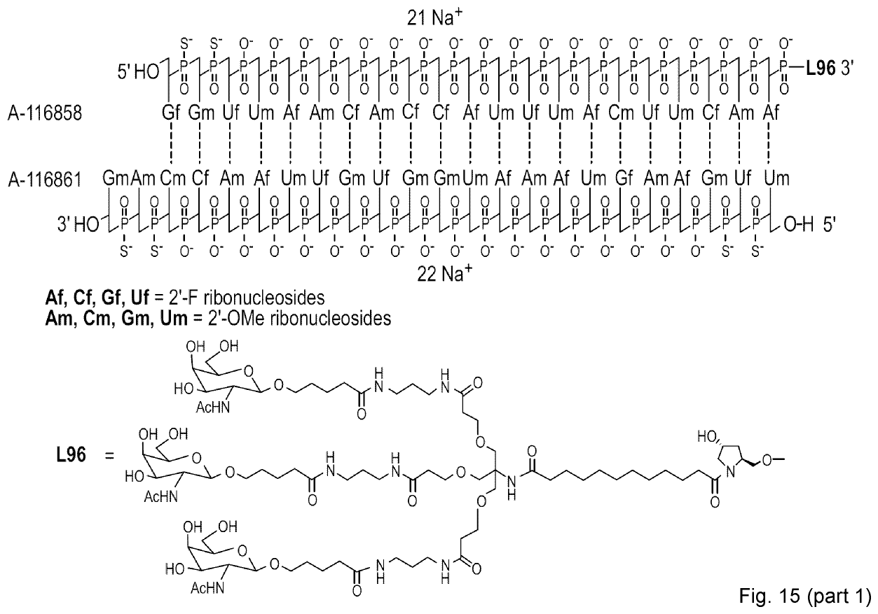

FIG. 14 illustrates the needle shroud 400 and the guide unit 700 of FIG. 9 in a sixth state. In the sixth state of the needle shroud 400, the medicament delivery device 100 may be completely removed from the skin of the user. The needle shroud 400 may completely cover the needle 8. The guide pin 90 snaps into the dead end 99 of the first channel 96 such that the needle shroud 400 is fixedly engaged to the guide unit 700.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of pharmaceutical formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug reservoir” adapted for use with a drug delivery device. The drug reservoir 101a may be, e.g., a cartridge, syringe, reservoir, or other solid or flexible vessel (bag) configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20 °C), or refrigerated temperatures (e.g., from about −4 °C to about 4 °C). In some instances, the drug reservoir may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N-(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F (ab) and F(ab′) 2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in conjunction with various embodiments of the present invention include, for example, Fab fragments, F(ab′) 2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Further examples of APIs for the prophylaxis of hemophilia A or B, with or without inhibitors, include an siRNA targeting antithrombin. An example of an siRNA targeting antithrombin is fitusiran. The term “prophylaxis” and “prophylactic treatment” are used interchangeably herein

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, pharmaceutical formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014 (E). As described in ISO 11608-1: 2014 (E), needle-based injection systems may be broadly distinguished into multi-dose container systems and single-dose (with partial or full evacuation) container systems. The container may be a replaceable container or an integrated non-replaceable container.

As further described in ISO 11608-1: 2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### Fitusiran as the API for the Medicament in the Device

Fitusiran is a synthetic, chemically modified double-stranded small interfering RNA (siRNA) oligonucleotide covalently linked to a tri-antennary N-acetyl-galactosamine (GalNAc) ligand targeting AT3 mRNA in the liver, thereby suppressing the synthesis of antithrombin. See, e.g., Pasi et al., N Engl J Med. (2017) 377 (9): 819-28. The nucleosides in each strand of fitusiran are connected through either 3′-5′ phosphodiester or phosphorothioate linkages, thus forming the sugar-phosphate backbone of the oligonucleotide.

The sense strand and the antisense strand contain 21 and 23 nucleotides, respectively. The 3′ end of the sense strand is conjugated to the GalNAc containing moiety (referred to herein as L96) through a phosphodiester linkage. The sense strand contains two consecutive phosphorothioate linkages at its 5′ end. The antisense strand contains four phosphorothioate linkages, two at the 3′ end and two at the 5′ end. The 21 nucleotides of the sense strand hybridize with the complementary 21 nucleotides of the antisense strand, thus forming 21 nucleotide base pairs and a two-base overhang at the 3′-end of the antisense strand. See also U.S. Pat. Nos. 9,127,274, 11,091,759, US2020/0163987A1, and WO 2019/014187, the entire contents each of which are expressly incorporated herein by reference.

The two nucleotide strands of fitusiran are shown below:

sense strand:
(SEQ ID NO: 1)
5′Gf-ps-Gm-ps-Uf-Um-Af-Am-Cf-Am-Cf-Cf-Af-Um-Uf-Um-
Af-Cm-Uf-Um-Cf-Am-Af-L96 3′,
and
antisense strand:
(SEQ ID NO: 2)
5′ Um-ps-Uf-ps-Gm-Af-Am-Gf-Um-Af-Am-Af-Um-Gm-Gm-
Uf-Gm-Uf-Um-Af-Am-Cf-Cm-ps-Am-ps-Gm 3′,

wherein
Af=2′-deoxy-2′-fluoroadenosine
Cf=2′-deoxy-2′-fluorocytidine
Gf=2′-deoxy-2′-fluoroguanosine
Uf=2′-deoxy-2′-fluorouridine
Am=2′-O-methyladenosine
Cm=2′-O-methylcytidine
Gm=2′-O-methylguanosine
Um=2′-O-methyluridine
“-” (hyphen)=3′-5′ phosphodiester linkage sodium salt
“-ps-”=3′-5′ phosphorothioate linkage sodium salt
   and wherein L96 has the following formula:

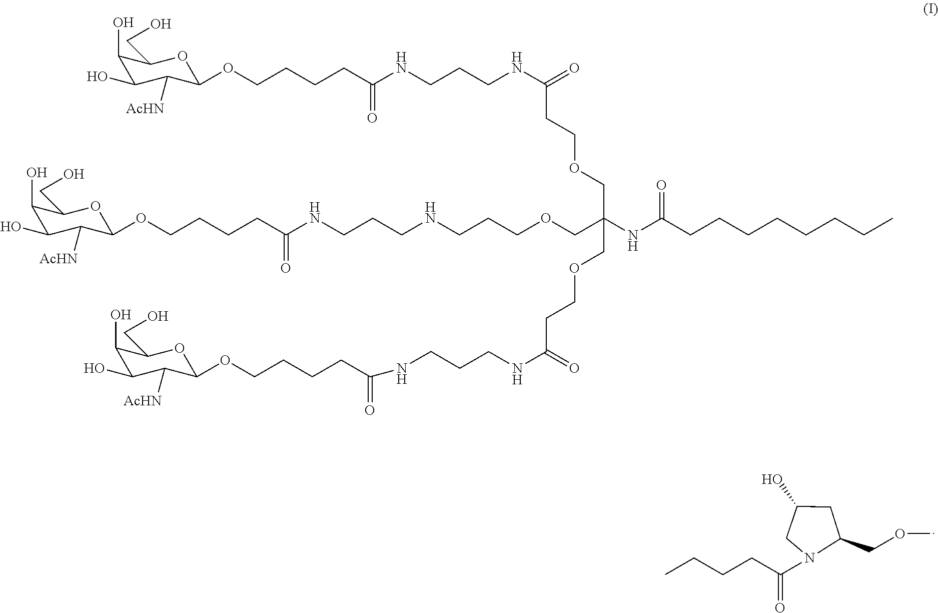

As used herein, the terms 2′-deoxy-2′-fluoroadenosine and 2′-fluoroadenosine may be used interchangeably. As used herein, the terms 2′-deoxy-2′-fluorocytidine and 2′-fluorocytidine may be used interchangeably. As used herein, the terms 2′-deoxy-2′-fluoroguanosine and 2′-fluoroguanosine may be used interchangeably. As used herein, the terms 2′-deoxy-2′-fluorouridine and 2′-fluorouridine may be used interchangeably.

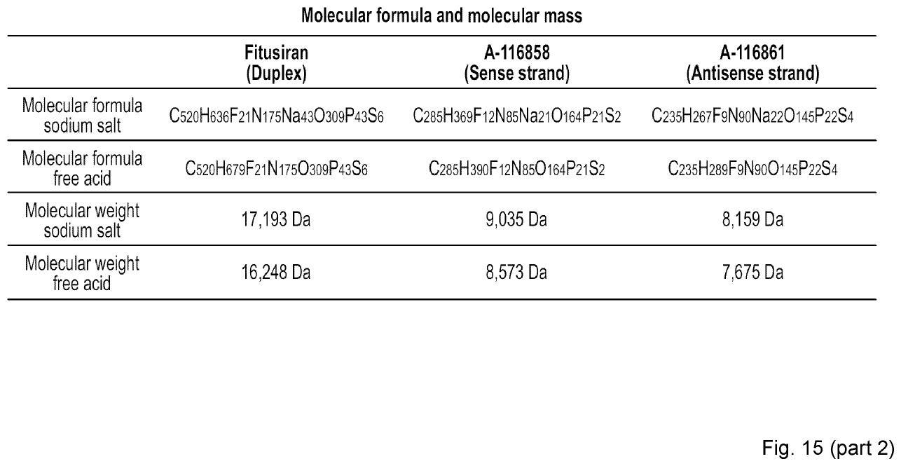

The expanded structural formula, molecular formula, and molecular weight of fitusiran are shown in FIG. 15.

The structure of fitusiran can also be described using the following diagram, wherein the X is O:

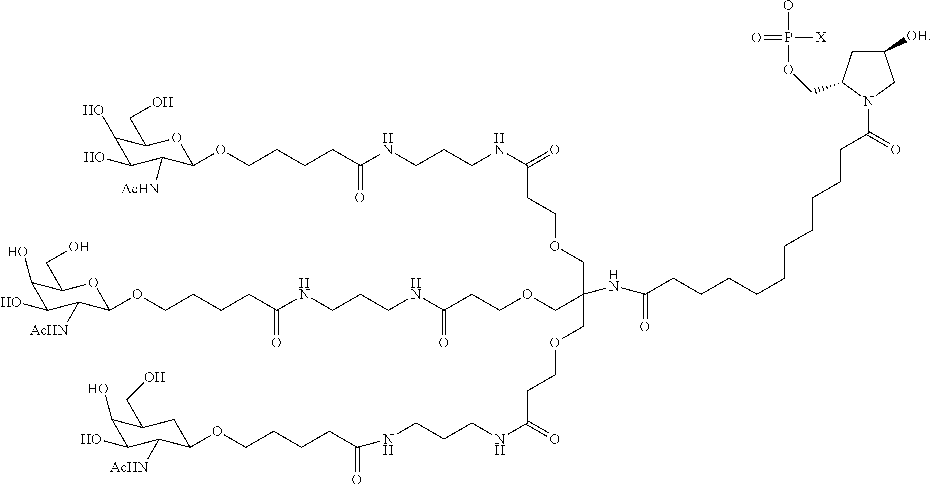

Fitusiran is shown in FIG. 15 in sodium salt form.

In some embodiments, the device delivers fitusiran in an aqueous solution, wherein fitusiran is at a concentration of about 40 to about 200 mg/mL (e.g., about 50 to about 150 mg/mL, about 80 to about 110 mg/mL, or about 90 to about 110 mg/mL). As used herein, values intermediate to recited ranges and values are also intended to be part of this disclosure. In addition, ranges of values using a combination of any of recited values as upper and/or lower limits are intended to be included. In further embodiments, the pharmaceutical formulation comprises fitusiran in an aqueous solution at a concentration of about 40, about 50, about 75, about 100, about 125, about 150, or about 200 mg/mL. In certain embodiments, fitusiran is provided in an aqueous solution at a concentration of about 100 mg/mL.

The term “deliver,” “delivers,” or “delivering” is intended to mean “administer,” “administers,” or “administering.”

Unless specifically stated or otherwise evident from the context, as used herein, the term “approximately” or “about” refers to a value that is within an acceptable error range for a particular value determined by a person of ordinary skill, a portion of which will depend on how the measurement or determination is made. For example, “approximately” or “about” may mean a range of up to 10% (ie, ±10%). Therefore, “approximately” or “about” can be understood as greater than or less than 10%, 9%, 8%, 7%, 6%, 5%, 4%, 3%, 2%, 1%, 0.5%, 0.1%, 0.05%, 0.01%, or 0.001%. When a specific value is provided in this disclosure, unless otherwise stated, the meaning of “approximately” or “about” should be assumed to be within an acceptable error range for that specific value.

While the fitusiran dosage weight described herein refers to the weight of fitusiran free acid (active moiety), administration of fitusiran to patients herein refers to administration of fitusiran sodium (drug substance) provided in a pharmaceutically suitable aqueous solution (e.g., a phosphate-buffered saline at a physiological pH). For example, about 100 mg/mL fitusiran means about 100 mg of fitusiran free acid (equivalent to about 106 mg fitusiran sodium, the drug substance) per mL. Unless otherwise indicated, a fitusiran weight recited in the present disclosure is the weight of fitusiran free acid (the active moiety).

In some embodiments, a pharmaceutical formulation in the device comprises fitusiran in a phosphate-buffered saline. The phosphate concentration in the solution may be about 1 to about 10 mM (e.g., about 2, about 3, about 4, about 5, about 6, about 7, about 8, or about 9 mM), with a pH of about 6.0-8.0. The pharmaceutical formulations herein may include a stabilizing agent such as EDTA. The pharmaceutical formulations may be preservative-free. In some embodiments, the fitusiran pharmaceutical formulation in the device is preservative-free and comprises, consists of, or consists essentially of about 100 mg of fitusiran per mL of an approximately 5 mM phosphate buffered saline (PBS) solution. In some embodiments, the fitusiran pharmaceutical formulation in the device is preservative-free and comprises, consists of, or consists essentially of fitusiran in an approximately 5 mM phosphate buffered saline (PBS) solution. The PBS solution is composed of sodium chloride, dibasic sodium phosphate (heptahydrate), and monobasic sodium phosphate (monohydrate). Sodium hydroxide solution and diluted phosphoric acid may be used to adjust the pH of the pharmaceutical formulation to about 7.0 or about 7.1.

In some embodiments, the fitusiran pharmaceutical formulation in the device for subcutaneous delivery contains fitusiran in a 5 mM phosphate buffered saline having 0.64 mM NaH₂PO₄, 4.36 mM Na₂HPO₄, and 84 mM NaCl at pH 7.0. In certain embodiments, the pharmaceutical formulation of fitusiran solution for subcutaneous delivery is shown in Table 1 below:

TABLE 1
Exemplary Fitusiran Pharmaceutical Formulation

Pharmaceutical Formulation

| | Percentage | Per ml |
|---|---|---|
| Components | [%] | [mg] |
| Fitusiran (active moiety) | 10 | 100 |
| [equivalent to fitusiran sodium] | | [106] |
| Sodium chloride | 0.49 | 4.909 |
| Dibasic sodium phosphate | 0.12 | 1.169 |
| (heptahydrate) | | |
| Monobasic sodium phosphate | <0.01 | 0.0885 |
| (monohydrate) | | |
| Phosphoric acid, concentrated | — | q.s. pH 7.0 |
| Sodium hydroxide | — | q.s. pH 7.0 |
| Water for subcutaneous | q.s. 100 | q.s. 1 mL |
| delivery | | |

*q.s.: quantum satis

In some embodiments, the pharmaceutical formulation of fitusiran solution for subcutaneous delivery with the device can be described as shown in Table 2 below.

TABLE 2
Exemplary Fitusiran Pharmaceutical Formulation

| | Pharmaceutical Formulation |
|---|---|
| Components | (mg) |
| Fitusiran (active moiety) | 100 |
| [equivalent to fitusiran sodium] | [106] |
| NaH₂PO₄*H₂O | 0.0885 |
| Na₂HPO₄*7H₂O | 1.169 |
| NaCl | 4.909 |
| 0.1N NaOH | q.s. |
| 0.1M H₃PO₄ | q.s. |
| Purified water | Ad 1 mL |

In some embodiments, the device may be used to deliver a single dose of fitusiran wherein the single dose comprises about 20 to about 80 mg of fitusiran (e.g., about 20 mg, about 25 mg, about 30 mg, about 40 mg, about 50 mg, or about 80 mg). In some embodiments, the device may be used to deliver single dose of fitusiran, wherein the single dose comprises about 1 to about 30 mg of fitusiran (e.g., about 1.25 mg, about 2.5 mg, about 5 mg, about 10 mg, about 20 mg, or about 30 mg).

In one embodiment, the device may be used to deliver a single dose of about 80 mg of fitusiran. In one embodiment, the device may be used to deliver a single dose of about 50 mg of fitusiran. In one embodiment, the device may be used to deliver a single dose of about 20 mg of fitusiran. In one embodiment, the device may be used to deliver a single dose of about 30 mg of fitusiran. In one embodiment, the device may be used to deliver a single dose of about 10 mg of fitusiran. In one embodiment, the device may be used to deliver a single dose of about 5 mg of fitusiran. In one embodiment, the device may be used to deliver a single dose of about 2.5 mg of fitusiran. In one embodiment, the device may be used to deliver a single dose of about 1.25 mg of fitusiran.

In some embodiments, the single dose of fitusiran may be delivered in about 0.5 mL to about 1 mL delivery volumes (e.g., about 0.5 mL, about 0.6 mL, about 0.7 mL, about 0.8 mL, about 0.9 mL, or about 1 mL). Other delivery volumes described herein may also be used.

In one embodiment, the device may be used to deliver a single dose of about 80 mg of fitusiran in about 0.8 mL (about 100 mg fitusiran/mL). In one embodiment, the device may be used to deliver a single dose of about 50 mg of fitusiran in about 0.5 mL (about 100 mg fitusiran/mL). In one embodiment, the device may be used to deliver a single dose of about 20 mg of fitusiran in about 0.5 mL (about 40 mg fitusiran/mL). In one embodiment, the device may be used to deliver a single dose of about 30 mg of fitusiran in about 0.5 mL (about 60 mg fitusiran/mL). In one embodiment, the device may be used to deliver a single dose of about 10 mg of fitusiran in about 0.5 mL (about 20 mg fitusiran/mL). In one embodiment, the device may be used to deliver a single dose of about 5 mg of fitusiran in about 0.5 mL (about 10 mg fitusiran/mL). In one embodiment, the device may be used to deliver a single dose of about 2.5 mg of fitusiran in about 0.5 mL (about 5 mg fitusiran/mL). In one embodiment, the device may be used to deliver a single dose of about 1.25 mg of fitusiran in about 0.5 mL (about 2.5 mg fitusiran/mL).

In one embodiment, the device delivers fitusiran at a prophylactically effective amount to prophylactically treat hemophilia (e.g., hemophilia A or B, in a patient with or without inhibitors) in a patient in need thereof (e.g., a hemophilia A or B patient, with or without inhibitors). “Prophylactically effective amount” refers to the amount of fitusiran that helps the patient with hemophilia A or B, with or without inhibitors to achieve a desired clinical endpoint such as reducing the Annualized Bleeding Rate (ABR), Annualized Joint Bleeding Rate (AjBR), Annualized Spontaneous Bleeding Rate (AsBR), or the frequency of bleeding episodes. As used herein in the context of fitusiran, the term “treat” “treating,” or “treatment” includes prophylactic treatment of the disease and refers to achievement of a desired clinical endpoint.

A hemophilia A or B patient with inhibitors refers to a patient who has developed alloantibodies to the factor he/she has previously received (e.g., factor VIII for hemophilia A patients or factor IX for hemophilia B patients). A hemophilia A or B patient with inhibitors may become refractory to replacement coagulation factor therapies. A patient without inhibitors refers to a patient who does not have such alloantibodies. The present treatment methods may be beneficial for hemophilia A patients with inhibitors, as well as for hemophilia B patients with inhibitors.

As used herein, a patient with “hemophilia A or B, with or without inhibitors,” or refers to 1) a hemophilia A patient with inhibitors, or 2) a hemophilia B patient with inhibitors, 3) a hemophilia A patient without inhibitors, or 4) a hemophilia B patient without inhibitors. As used herein, a patient refers to a human patient. A patient can also refer to a human subject.

In some embodiments, the device may be used to prophylactically treat a patient with hemophilia A or B, with or without inhibitors, with a subcutaneous dose of about 50 mg of fitusiran once every two months (or every eight weeks). In other embodiments, the device may be used to prophylactically treat a patient with hemophilia A or B, with or without inhibitors, with a subcutaneous dose of about 50 mg of fitusiran every month (or every four weeks). In yet other embodiments, the device may be used to prophylactically treat a patient with hemophilia A or B, with or without inhibitors, with a subcutaneous dose of about 80 mg of fitusiran every two months (or every eight weeks). In yet other embodiments, the device may be used to prophylactically treat a patient with hemophilia A or B, with or without inhibitors, with a subcutaneous dose of about 80 mg of fitusiran every month (or every four weeks). In yet other embodiments, the device may be used to prophylactically treat a patient with hemophilia A or B, with or without inhibitors, with a subcutaneous dose of about 20 mg of fitusiran every two months (or every eight weeks). In yet other embodiments, the device may be used to prophylactically treat a patient with hemophilia A or B, with or without inhibitors, with a subcutaneous dose of about 20 mg of fitusiran every month (or every four weeks). In yet other embodiments, the device may be used to prophylactically treat a patient with hemophilia A or B, with or without inhibitors, with a subcutaneous dose of about 10 mg of fitusiran every month (or every four weeks). In yet other embodiments, the device may be used to prophylactically treat a patient with hemophilia A or B, with or without inhibitors, with a subcutaneous dose of fitusiran at about 30 mg every month (or every four weeks). In yet other embodiments, the device may be used to prophylactically treat a patient with hemophilia A or B, with or without inhibitors, with a subcutaneous dose of fitusiran at about 5 mg every month (or every four weeks). In yet other embodiments, the device may be used to prophylactically treat a patient with hemophilia A or B, with or without inhibitors, with a subcutaneous dose of fitusiran at about 2.5 mg every month (or every four weeks). In yet other embodiments, the device may be used to prophylactically treat a patient with hemophilia A or B, with or without inhibitors, with a subcutaneous dose of fitusiran at about 1.25 mg every month (or every four weeks).

Accordingly, provided herein is a method of prophylactic treatment of a patient with hemophilia A or hemophilia B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of prophylactic treatment of a patient with hemophilia A or hemophilia B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Further provided herein is a method of reducing the frequency of bleeding episodes in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the frequency of bleeding episodes in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the ABR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the ABR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AjBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). The fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AjBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

Also, provided herein is a method of reducing the AsBR in a patient with hemophilia A or B, with or without inhibitors, comprising subcutaneously delivering with the device a prophylactically effective amount of fitusiran to the patient in need thereof. The prophylactically effective amount of fitusiran may be any dose provided herein, such as about 1 to about 80 mg, about 1 to about 30 mg, or about 20 to about 80 mg. The prophylactically effective amount of fitusiran may be, for example, about 1.25 mg, about 2.5 mg, about 5 mg, about 25 mg, about 30 mg, about 50 mg, or about 80 mg. The prophylactically effective amount of fitusiran may be delivered every month (or every four weeks) or once every two months (or every eight weeks). Fitusiran may be delivered in about 0.5 mL to about 1 mL delivery volumes (e.g., about 0.5 mL, about 0.6 mL, about 0.7 mL, about 0.8 mL, about 0.9 mL, or about 1 mL).

As an example, a method of reducing the AsBR in a patient with hemophilia A or B, with or without inhibitors, may comprise subcutaneously delivering with the device about 50 mg of fitusiran to the patient in need thereof every month (or every four weeks) or once every two months (or every eight weeks). The about 50 mg of fitusiran may be delivered in about 0.5 mL PBS (at a concentration of about 100 mg fitusiran/mL).

### REFERENCE NUMERALS

2 reservoir body
3 retainer
4 stopper
5 drive element
7, 7′ activation element
8 needle
10, 10′ self-driven medicament container
21 drive compartment
22 medicament compartment
23 outlet
24 needle shield
31, 31′ opening
32 guide element
61, 61′ shaft
62 flexible arm
62′ stopper bar
63 contact element
64 inclined surface
71 engagement feature
72 guide groove
90 guide pin
92 torsion protection
94 flexible bars
96 first channel
97 second channel
98 bent
99 dead end
100 medicament delivery device
102 barb
200 housing
210 distal end wall
211 opening
212 protrusion
221 blocker arm
300 outer button
310 lock protrusion
400 needle shroud
410 opening
420 proximal inner surface
430 proximal end
500 needle shroud spring
600 end cap
700 guide unit

1-19. (canceled)

20. A self-driven medicament container for storing and discharging a medicament in response to a user interaction, the self-driven medicament container comprising:
a reservoir body;
a retainer;
a stopper configured to divide an interior of the reservoir body into a drive compartment and a medicament compartment, the medicament compartment containing the medicament and comprising an outlet through which the medicament can be discharged in a dispensing operation;
a drive element;
a plunger having a shaft with a locking element configured to engage the retainer to prevent a distal movement of the plunger relative to the reservoir body and a contact element configured to contact the stopper; and
a release mechanism, configured to disengage the locking element from the retainer in response to the user interaction thereby enabling a distal movement of the plunger relative to the reservoir body,
wherein the medicament compartment is distal to the drive compartment;
wherein the drive element is positioned in the drive compartment before and after the locking element is disengaged from the retainer;
wherein the stopper is further configured to seal the medicament compartment relative to the drive compartment; and
wherein the drive element is configured to provide an axial force to the plunger in order to move the plunger distally relative to the reservoir body thereby moving the stopper relative to the reservoir body and discharging the medicament, when the locking element is disengaged from the retainer.

21. The self-driven medicament container of claim 20, wherein the reservoir body has an interior wall surface that is in contact with the medicament, the interior wall surface defining an interior cross-section of the reservoir body.

22. The self-driven medicament container of claim 21, wherein the interior cross-section of the reservoir body is constant at least along a length of the reservoir body along which the stopper is moved during the dispensing operation.

23. The self-driven medicament container of claim 21, wherein the interior wall surface of the reservoir body is configured to be continuously connected to an outer surface of the self-driven medicament container.

24. The self-driven medicament container of claim 21, wherein the interior cross-section is circular.

25. The self-driven medicament container of claim 20, wherein the drive element is a spring.

26. The self-driven medicament container of claim 20, wherein the release mechanism comprises an activation element which is configured to disengage the locking element from the retainer when performing an activation movement, wherein the activation movement comprises an axial and/or rotational movement of the activation element relative to the reservoir body.

27. The self-driven medicament container of claim 20, wherein the shaft extends into the reservoir body through an opening in the retainer such that the locking element is proximal to the retainer before the disengagement, and wherein the opening is configured such that the locking element can pass through the retainer when the locking element has been disengaged from the retainer.

28. The self-driven medicament container of claim 26, wherein an interface can be established between an engagement feature which is formed on one of the locking element and the activation element and an inclined surface which is provided on the other one of the locking element and the activation element, wherein, when the interface is established and the engagement feature moves relative to the inclined surface, e.g. the engagement feature slides along the inclined surface, the locking element is disengaged from the retainer.

29. The self-driven medicament container of claim 26, wherein the locking element is formed by two or more flexible arms that are configured to radially deflect in response to a movement of the activation element relative to the reservoir body.

30. The self-driven medicament container of claim 29, wherein the flexible arms are evenly distributed in a circumferential direction.

31. The self-driven medicament container of claim 26, wherein the locking element is formed by a stopper bar which is configured to abut a proximal surface of the retainer before the user interaction, and wherein the locking element is configured to rotate relatively to the retainer in order to align with the opening of the retainer in response to the user interaction such that the locking element can pass through the opening in a distal direction.

32. The self-driven medicament container of claim 20, wherein the retainer is mounted to a proximal end of the reservoir body or to an inner wall of the reservoir body.

33. The self-driven medicament container of claim 20, wherein the drive element is completely contained in the drive compartment of the reservoir body.

34. The self-driven medicament container of claim 20, wherein a distally facing end surface of the contact element is a closed surface configured to contact the stopper over its full cross-section.

35. The self-driven medicament container of claim 20, wherein a longitudinal axis of the shaft is parallel to a longitudinal axis of the reservoir body before disengagement of the locking element from the retainer.

36. A medicament delivery device comprising:
a self-driven medicament container according to claim 20,
a needle connected to the outlet;
an elongated housing which the self-driven medicament container abuts, wherein the elongated housing has a distal end wall having an opening through which the needle protrudes distally beyond the housing; and
an outer button connected to a proximal portion of the housing and configured to interact with the release mechanism of the self-driven medicament container such that the locking element is disengaged from the retainer when the outer button is moved distally relative to the housing.

37. The medicament delivery device of claim 36, further comprising:
a needle shroud movably connected to the housing and protruding distally beyond a tip of the needle in a first position;
wherein the housing comprises a button lock configured to engage with the outer button to block a distal movement of the outer button relative to the housing when the needle shroud is in its first position and configured to disengage from the outer button when the needle shroud is moved proximally relative to the housing to a second position.

38. The medicament delivery device of claim 37, wherein the button lock comprises at least two blocker arms that are configured to deflect radially outwardly due to an interaction with the needle shroud, when the needle shroud is moved proximally relative to the housing from its first position to its second position.

39. A method of delivering a medicament from a medicament delivery device, the method comprising
providing a medicament delivery device, comprising:
a self-driven medicament container, for storing and discharging a medicament in response to a user interaction, the self-driven medicament container comprising:
a reservoir body;
a retainer;
a stopper configured to divide an interior of the reservoir body into a drive compartment and a medicament compartment, the medicament compartment containing the medicament and comprising an outlet through which the medicament can be discharged in a dispensing operation; and
a drive element;
a plunger having a shaft with a locking element configured to engage the retainer to prevent a distal movement of the plunger relative to the reservoir body and a contact element configured to contact the stopper; and
a release mechanism, configured to disengage the locking element from the retainer in response to the user interaction thereby enabling a distal movement of the plunger relative to the reservoir body,
wherein the medicament compartment is distal to the drive compartment;
wherein the drive element is positioned in the drive compartment before and after the locking element is disengaged from the retainer;
wherein the stopper is further configured to seal the medicament compartment relative to the drive compartment; and
wherein the drive element is configured to provide an axial force to the plunger in order to move the plunger distally relative to the reservoir body thereby moving the stopper relative to the reservoir body and discharging the medicament, when the locking element is disengaged from the retainer;
a needle connected to the outlet;
an elongated housing which the self-driven medicament container abuts, wherein the elongated housing has a distal end wall having an opening through which the needle protrudes distally beyond the housing; and
an outer button connected to a proximal portion of the housing and configured to interact with the release mechanism of the self-driven medicament container such that the locking element is disengaged from the retainer when the outer button is moved distally relative to the housing, and
causing the drive element to provide the axial force to the plunger.
